(19) United States
(12) Patent Application Publication
(10) Pub. No.: US 2007/0154916 A1
(43) Pub. Date: Jul. 5, 2007

(54) Method for detecting cancer and a method for suppressing cancer

(76) Inventors: Johji Inazawa, Tokyo (JP); Issei Imoto, Tokyo (JP); Jun Inoue, Tokyo (JP); Akiko Furihata, Tokyo (JP); Sana Yokoi, Tokyo (JP); Itaru Sonoda, Tokyo (JP); Hideaki Tanami, Tokyo (JP); Hiroyuki Izumi, Tokyo (JP); Kuniyasu Saigusa, Tokyo (JP); Shin Hayashi, Tokyo (JP); Hisashi Takada, Tokyo (JP); Ayako Suzuki, Tokyo (JP)

Correspondence Address: BIRCH STEWART KOLASCH & BIRCH, PO BOX 747, FALLS CHURCH, VA, 22040-0747, US

(21) Appl. No.: 11/591,486

(22) Filed: Nov. 2, 2006

(30) Foreign Application Priority Data
Nov. 4, 2005 (JP) 2005-304497

(51) Int. Cl. C12Q 1/68; C12P 19/34; A61K 48/00

(52) U.S. Cl. 435/6; 435/91.2; 514/44; 977/924

(57) ABSTRACT

An object of the invention is to find a cancer-associated gene to be used as an index for detecting canceration of cells and degree of malignancy of cancer, so as to to provide a method for detecting cancer using the cancer-associated gene as an index and provide a method of suppressing/treating cancer using the cancer-associated gene as essential part. According to the present invention, specific genes which are amplified or deleted in myeloma as compared with normal cell have been collectively found, and a method for detecting cancer using amplification or deletion of these cancer-associated genes as an index is provided. Further, cancer can be suppressed by introducing a gene which is deleted in cancer cells amond these cancer-associated genes into cancer and inhibiting the transcription product of the gene amplified.

### TECHNICAL FIELD

The present invention relates to a method of detecting canceration and malignancy of cancer using a specific cancer-associated gene as an index, and also relates to a method of suppressing/treating cancer using a specific cancer-associated gene as essential part.

### BACKGROUND ART

A mortality rate of cancer is presently the top end in Japan and occupies one third of the total mortality causes. The mortality rate of cancer goes on increasing and is predicted to occupy about 50% in 10 years. It has been elucidated that cancer is caused and aggravated due to accumulation of abnormalities of many genes. It has been reported that acceleration of oncogene expression and deceleration of cancer suppressor gene expression due to deletion are involved in canceration. Furthermore, it is also known that abnormalities of a gene directly involved in cell differentiation and proliferation and a gene involved in a DNA repair system are involved in canceration.

However, studies that have been hitherto conducted are not sufficient to explain the canceration mechanism in cancer patients. A group of genes involved in canceration varies depending upon the type of cancer. Furthermore, since the individual characters of cancers differ even if they belong to the same type, it has been difficult to systematically analyze the abnormality of which gene group causes cancer. Therefore, it cannot be said that a sufficient diagnostic method for the initial state of cancer and a sufficient diagnostic means for checking degree of malignancy of cancer based on genomic analysis of cancer cells have been provided.

### DISCLOSURE OF THE INVENTION

An object of the invention is to find a cancer-associated gene to be used as an index for detecting canceration of cells and degree of malignancy of cancer and to provide a method for detecting cancer using the cancer-associated gene as an index. Another object of the present invention is to provide a method of suppressing/treating cancer using the cancer-associated gene as essential part.

Generally, when a chromosomal abnormality takes place, the cell causes apoptosis to death. Therefore, proliferation of an abnormal cell does not occur in mechanism. However, in some cases, a cell having a chromosomal abnormality may happen to initiate proliferation for an unknown reason through a loophole of the biological control mechanism that should be strictly controlled, thus initiating canceration. Therefore, amplification and deletion of a genome at a chromosomal level are critical causes of canceration. In the case of amplification, expression of a gene present in the amplified genomic region is accelerated, whereas, in the case of deletion, the expression level of a gene present in the deleted genomic region is significantly decelerated. When such abnormalities are accumulated, a cell may probably cause unregulated proliferation.

Comparative genomic hybridization (CGH) is a simple and quick method, that is, the best method, for analyzing gene abnormalities associated with genomic amplification and deletion of a plurality of genes. To analyze abnormality of a gene on the genome involved in canceration and malignant alteration of cancer, it is extremely important to select a group of genes to be printed on a CGH microarray.

The present inventors screened a group of highly potential genes that may be involved in canceration from the databases “National Cancer for Biotechnology” and “University of California Santa Cruz Biotechnology.” They further subjected the DNA thus screened to BLAST search to select genes that conceivably play an important role in the onset of cancer.

BAC/PAC clones containing these candidate cancer-associated genes are carefully selected and individually amplified (inexhaustibly amplified). Then, about 800 types of clones thus amplified were loaded on a substrate to form a “MCG cancer array” substrate (hereinafter also referred to “MCG cancer array”). The present invention encompasses the MCG cancer array within its technical range.

The present inventors found cancer-associated genes to be used as cancer detection indexes in several types of cancer by use of the MCG cancer array. Based on the finding, they accomplished one of the present inventions.

More specifically, the present invention provides a method of detecting (hereinafter referred to also as “the detection method of the invention”) cancer using a specific cancer-associated gene as an index. Also in the present invention, there is provided a means for suppressing/treating cancer using the cancer-associated gene. More specifically, the present invention provides a means for suppressing/treating cancer by introducing a specific deletion cancer-associated gene into a cancer cell and a means for suppressing/treating cancer by inhibiting the function of the transcriptional product (mRNA) of a specific amplification cancer-associated gene. These means for suppressing/treating cancer will be explained later.

The present invention provides a method for detecting myeloma, wherein canceration of a specimen is detected based on an index of not less than 1.5 fold amplification of at least one gene selected from the group consisting of
BCL9 gene, MCL1 gene, AF1Q gene, MUC1 gene, DAP3 gene, ARHGEF2 gene, PMF1 gene, BRAL1 gene, PRCC gene, TRK gene, NTRK1 gene, CRP gene, ATF6 gene, PBX1 gene, ABL2 gene, LAMC2 gene, TP gene, ABL gene, CAN gene, KRAS2 gene, KRAG gene, PTHLH gene, NCOR1 gene, ZNF287 gene, D17S128 gene, BCL2 gene, FVT1 gene, and PI5 gene;
   in the specimen in comparison with a normal cell.

The present invention further provides a method for detecting myeloma according to the present invention as mentioned above, wherein canceration of a specimen is detected based on an index of not less than 4 fold amplification of at least one gene selected from the group consisting of
ABL gene, CAN gene, KRAS2 gene, KRAG gene, PTHLH gene, NCOR1 gene, ZNF287 gene, D17S128 gene, BCL2 gene, FVT1 gene, and PI5 gene;
   in the specimen in comparison with a normal cell.

The present invention further provides a method for detecting myeloma, wherein canceration of a specimen is detected based on an index of a heterozygous deletion of at least one gene selected from the group consisting of
TGFβR3 gene, ABCD3 gene, ZNF198 gene, FGF9 gene, FLT1 gene, BRCA2 gene, FKHR gene, TPT1 gene, LCP1 gene, RB1 gene, DACH gene, PIBF1 gene, KLF12 gene, GPC5 gene, GPC5 (2) gene, GPC6 gene, ABCC4 gene, RAP2A gene, FGF14 gene, ERCC5 gene, EFNB2 gene, ING1 gene, TFDP1 gene, GAS6 gene, D13S327 gene, TEP1 gene, MMP14 gene, BCL2L2 gene, FKHL1 gene, MBIP gene, HNF3A gene, SSTR1 gene, PNN (DRS) gene, CDKN3 gene, RBBP1 gene, DAAM1 gene, MNAT1 gene, HIF1A gene, ESR2 gene, MAX gene, EIF2S1 gene, RAD51L1 gene, IGHG1 gene, HSXIAPAF1 gene, MN1 gene, TSPY gene, EPS15 gene, PGR gene, YAP1 gene, cIAP1 gene, MMP7 gene, DYNEIN gene, ETS1 gene, and FLI1 gene.
   in the specimen.

The present invention further provides a method for detecting myeloma, wherein canceration of a specimen is detected based on an index of a homozygous deletion of at least one gene selected from the group consisting of
EPS15 gene, PGR gene, YAP1 gene, cIAP1 gene, MMP7 gene, DYNEIN gene, ETS1 gene, FLI1 gene, IGHG1 gene, and TSPY gene;
   in the specimen.

Preferably in the above, the detection is performed by a CGH method, DNA chip method, quantitative PCR method or real time RT-PCR method.

Preferably in the above, detection is performed by a CGH method or DNA chip method and a plurality of types of DNA fragments to be fixed onto the detection substrate are genomic DNA, cDNA or synthetic oligonucleotides.

Preferably in the above, the detection is performed by a CGH method, and a plurality of types of DNA fragments to be fixed onto the detection substrate are genomic DNA, and the genomic DNA is a gene amplification product of BAC DNA, YAC DNA or PAC DNA.

The present invention further provides a method for suppressing a myeloma cell, which comprises introducing a gene, whose deletion is involved in canceration of a myeloma cell, into a myeloma cell.

The present invention further provides a method for suppressing a myeloma cell, which comprises introducing at least one gene selected from the group consisting of EPS15 gene, PGR gene, cIAP1 gene, DYNEIN gene, YAP1 gene, MMP7 gene, ETS1 gene, FLI1 gene, IGHG1 gene and TSPY gene into a myeloma cell.

The present invention further provides a method of suppressing a myeloma cell, which comprises applying, to a myeloma cell, a nucleic acid antagonizing a transcriptional product of a gene whose amplification is involved in canceration of the myeloma cell.

The present invention further provides a method of suppressing a myeloma cell, which comprises applying, to a myeloma cell, a nucleic acid antagonizing a transcriptional product of at least one gene selected from the group consisting of ABL gene, CAN gene, KRAS2 gene, ZNF285 gene, D17S128 gene, BCL2 gene, KRAG gene, PTHLH gene, NCOR1 gene, FVT1 gene and PI5 gene.

Preferably, the nucleic acid antagonizing a transcriptional product of a gene is small interference RNA against a transcriptional poroduct mRNA, or an antisense oligonucleotide of the mRNA.

### BRIEF DESCRIPTION OF THE DRAWINGS

FIG. 1 shows an illustration of genome analysis for a normal diploid cell by use of the MCG cancer array.

FIG. 2 shows a graph showing the results of the genome analysis for a cancer cell by use of the MCG cancer array.

### BEST MODE FOR CARRYING OUT THE INVENTION

A. The Detection Method of the Invention

The detection according to the invention may be carried out by CGH method, DNA chip method, quantitative PCR method, or real time RT-PCR method. To detect amplification or deletion of a gene, the DNA chip method or CGH method is preferably used and the CGH method is particularly preferable. When the expression of a cancer suppressor gene (corresponding to the “deletion gene” mentioned above) is suppressed by another cause except for gene deletion, such as acceleration of methylation of a CpG island of the gene and deceleration of acetylation of a protein associated with the gene, it is preferable to employ a detection means for detecting an transcriptional product of the gene, such as the real time RT-PCR method and the DNA chip method, capable of quantifying the transcribed product of the gene.

The specimen to be subjected to the detection method of the present invention is derived from a subject and corresponds to the type of cancer to be detected. To explain more specifically, a bone marrow biopsy specimen is used when a subject is checked for myeloma.

As a preferable embodiment of the detection method of the present invention, mention may be made of application of a CGH method to a substrate on which a plurality of types of gene amplification products having a specific genome DNA region obtained from a BAC (bacterial artificial chromosome) DNA, YAC (yeast artificial chromosome) DNA, or PAC (phage artificial chromosome) DNA are individually and separately fixed. In this embodiment, amplification and deletion gene of a genomic DNA can be analyzed by the CGH method.

The amount of the BAC DNA generally obtained is too little to fix onto numerous substrates practically used as genomic DNA fixed substrates. Therefore, the DNA must be obtained as an amplified product of a gene (the amplification process of the gene is also called as “inexhaustible process”). In the inexhaustible process, BAC DNA etc., was digested with a 4-nucleotide recognition enzyme, such as RsaI, DpnI, or HaeIII, and then, an adapter was added to ligate the digested fragments. The adapter is an oligonucleotide formed of 10 to 30 nucleotdes and preferably 15 to 25 nucleotides. The double stranded chain has a complementary sequence. After annealing, the 3′ end of the oligonucleotide forming a smooth end must be phosphorylated. Then, using a primer having the same sequence as one of the oligonucleotides serving as the adaptor, amplification is performed by PCR (Polymerase Chain Reaction). In this manner, the inexhaustible process can be carried out. On the other hand, an aminated oligonucleotide having 50 to 70 nucleotides characteristic in each of the BAC DNA and the like may be used as a detection probe.

The inexhaustibly amplified BAC DNA or the like (the same in the embodiment genomic DNA, cDNA or synthetic oligonucleotide is used) is fixed onto a substrate, preferably a solid substrate, to manufacture a desired DNA fixed substrate.

Examples of the solid substrate include glass, plastic, membrane and a three-dimensional array. Preferably a glass substrate such as a slide glass is preferable. The solid substrate formed of such as glass is preferably coated by depositing poly-L-lysine, amino silane, gold, and aluminium thereon and applied by an amino group modified DNA immobilization surface treatment.

The concentration of the inexhaustibly amplified DNA mentioned above (the same in the embodiment genomic DNA, cDNA or synthetic oligonucleotide is used) to be spotted on the substrate is preferably 10 pg/μl to 5 μg/μl, and more preferably, 1 ng/μl to 200 ng/μl. The amount of the spot is preferably 1 nl to 1 μl, and more preferably, 10 nl to 100 nl. The size and shape of individual spots to be fixed on the substrate are not particularly limited; however, for example, may be a diameter of 0.002 to 0.5 mm and a circular to elliptic shape as viewed from the top. The thickness of dry spots is not particularly limited; however, may be 1 to 100 μm. The number of spots are not particularly limited; however, preferably 10 to 50,000, and more preferably 100 to 5,000. Each DNA may be spotted in the range of a singular spot to quadruplicated spots, and preferably duplicated or triplicated spots.

The dry spots may be prepared by spotting a plurality of spots of BAC DNA and the like (the same in the embodiment genomic DNA, cDNA or synthetic oligonucleotide is used) inexhaustibly amplified on a substrate by means of a spotter, and drying the spots. As the spotter, use may be made of an inkjet printer, pin array printer, and bubble-jet (registered trade mark) printer; however, an inkjet printer may be preferably used. More specifically, use may be made of GENESHOT (NGK insulators Ltd., Nagoya) and high-throughput inkjet delivery system SQ series (manufactured by Cartesian Technologies, USA), etc.

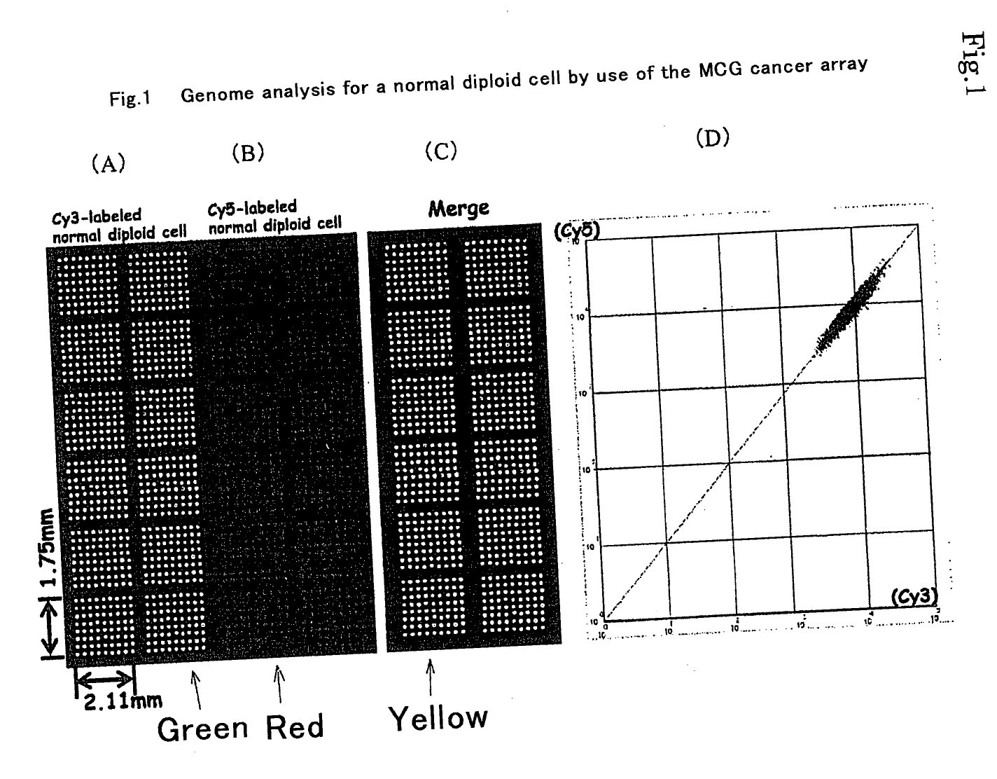

In the manner mentioned above, a desired DNA fixation substrate can be manufactured by fixing BAC DNA and the like (the same in the embodiment genomic DNA, cDNA or synthetic oligonucleotide is used) inexhaustibly amplified on a substrate, and preferably a solid substrate. Hybridization was actually performed using Cy-3 labeled genomic DNA derived from a normal diploid cell, and Cy-5 labeled genomic DNA derived from the same normal diploid cell separately on the MCG cancer array. The results are shown in FIG. 1, together with the hybridization results performed with the mixture of them (indicated by “Merge”). When Cy-3 labeled genomic DNA is used, green fluorescence is detected. When Cy-5 labeled genomic DNA is used, red fluorescence is detected. When both are mixed, yellow fluorescence is detected.

In the MCG cancer array shown in FIG. 1, 432 types of BAC DNA were printed. The BAC DNA collectively contains a group of cancer-associated genes such as oncogenes and cancer suppressor genes. In the one district of the array having 1.75 mm length and 2.11 wide, 72 DNA spots are printed. In total, 432 spots are arranged in a linear row and printed in duplicate. FIG. 1A shows the hybridization results of Cy-3 labeled normal diploid cell genomic DNA and thus all spots are green. FIG. 1B shows the hybridization results of Cy-5 labeled normal diploid cell genomic DNA and thus all spots are red. FIG. 1C (indicated “Merge” on the slide substrate) shows the hybridization results of a mixture of the Cy-3 labeled DNA and the Cy-5 labeled DNA and all spots are yellow. When the fluorescence intensity of Cy-3 is plotted on the transverse axis and that of Cy-5 is plotted on the vertical axis, all plots of signals draw a straight line and converged into an intensity of 5×10³ to 5×10⁴ (FIG. 1D).

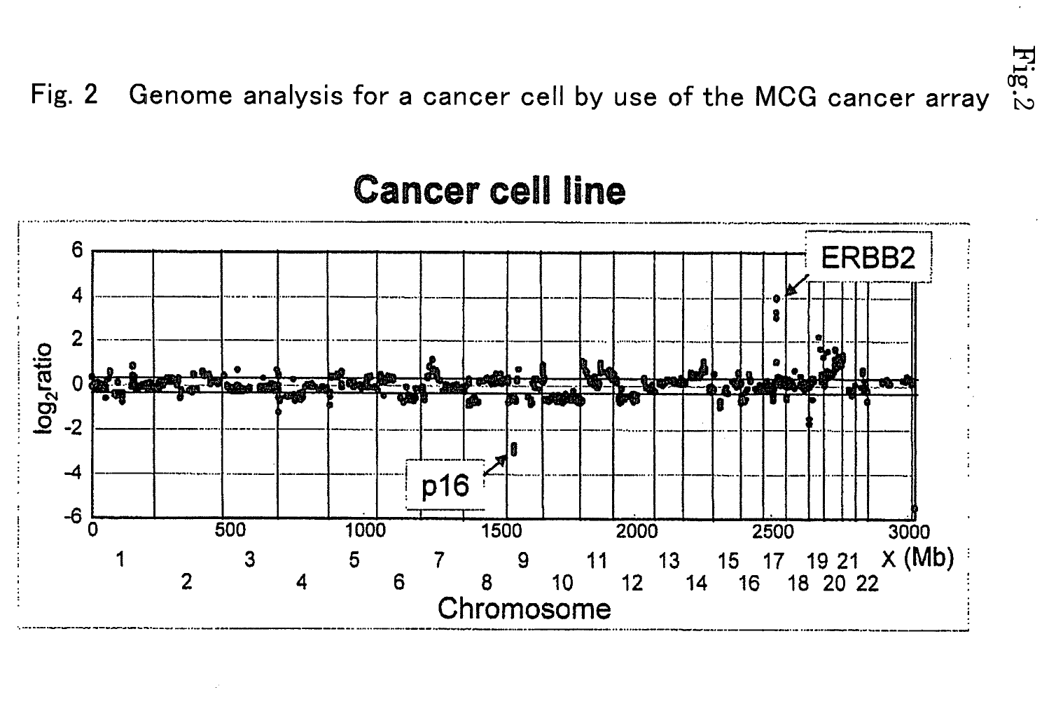

Furthermore, actually, DNA derived from a normal cell was labeled with Cy-5 and DNA derived from a cancer cell was labeled with Cy-3. They were subjected to comparative genomic hybridization. Data were taken in by a GenePix 4000B scanner. Individual pixels were analyzed and the results are shown in FIG. 2. The vertical axis of the graph in FIG. 2 is indicated by Log₂ Ratio and BAC clones having genomes from a short arm to a long arm of a chromosome are arranged on the transverse axis. The Cy-3 intensities of all spots are corrected to the same level as the Cy-5 intensities of all spots, and the ratio of Cy-3 intensity/Cy-5 intensity of each spot is obtained and a value of Log₂ Ratio is computationally obtained. BAC having a CDKN2A (p16) gene shows Log₂ Ratio=about −3 and Ratio=1/8, which clearly indicates a homozygous deletion. On the other hand, BAC having ERBB2 gene gives Log₂ Ratio=3-4 and Ratio=8-16, which demonstrates that ERBB2 genomic DNA is amplified 8 to 16 fold.

To identify a group of genes present in the chromosomal region amplified and deleted in a cancer cell by use of the MCG cancer array, genomic DNA derived from a healthy person and genomic DNA derived from a lung cancer cell are labeled with mutually different dye, for example, Cy-3 and Cy-5, in accordance with a customary method (for example, a nick translation method using dCTP). The labeling kits using the nick translation method using dCTP are sold by PanVera (Takara Shuzo Co., Ltd., a distributor in Japan) and Invitrogen (CA, USA). When the labeled DNA is hybridized with the DNA printed on the CGH array, it is more preferable to add Cot-1DNA, formamide, dextran sulfate, SSC (150 mM NaC1/15 mM sodium citrate), Yeast t-RNA, and SDS (sodium dodecyl sulfate). Furthermore, it is preferable to add a solution containing labeled DNA after it is denatured with heat. As a container for use in hybridization, a container that can be placed on a platform having a locking function and can bring a small amount of solution uniformly into contact with the array is preferable, and use of e.g., hybriman, is more preferable. The temperature of hybridization is preferably 30 to 70° C. and more preferably 38 to 45° C. The hybridization time is preferably 12 to 200 hours and more preferably 40 to 80 hours. The array can be washed with formamide, SSC solution or the like at room temperature. The washing of the array is an important step to reduce a nonspecific signal as much as possible. More preferably, the array was washed at room temperature, and then, washed with the same washing solution at 40 to 60° C., further washed in a solution containing SSC-SDS at 50° C., allowed to stand in a solution containing phosphate buffer/NP-40, and finally shaken in a solution containing SSC.

(1) Group of Genes Present in the Chromosome Amplified and Deleted in Myeloma

Using the MCG cancer array, a group of genes present in the chromosomal region amplified and deleted in a myeloma cell was identified. As a result of checking a gene amplified and having a Ratio value of 1.32 and or more, BCL9, MCL1, AF1Q, MUC1, DAP3, ARHGEF2, PMF1, BRAL1, PRCC, TRK, NTRK1, CRP, ATF6, PBX1, ABL2, LAMC2, and TP genes were detected. As a gene having a Ratio value of 4 or more, that is a gene amplified 4-fold or more than that of a normal cell gene, ABL, CAN, KRAS2, KRAG, PTHLH, NCOR1, ZNF287, D17S128, BCL2, FVT1, and PI5 genes were detected.

On the other hand, as a result of analyzing a deletion of a gene in a myeloma cell, as a gene having a Ratio value as low as 0.75 or less, that is, determined as a heterozygote, TGFβR3, ABCD3, ZNF198, FGF9, FLT3, FLT1, BRCA2, FKHR, TPT1, LCP1, RB1, DACH, PIBF1, KLF12, GPC5, GPC5 (2), GPC6, ABCC4, RAP2A, FGF14, ERCC5, EFNB2, ING1, TFDP1, GAS6, D13S327, TEP1, MMP14, BCL2L2, FKHL1, MBIP, HNF3A, SSTR1, PNN (DRS), CDKN3, RBBP1, DAAM1, MNAT1, HIF1A, ESR2, MAX, EIF2S1, RAD51L1, IGHG1, HSXIAPF1, MN1, and TSPY were detected. As a gene having a Ratio value as low as 0.25 or less, that is, having a homozygous deletion, EPS15, PGR, YAP1, cIAP1, MMP7, MMP1, DYNEIN, ETS1, FLI1, IGHG1, and TSPY genes were detected.

By checking the amplification and deletion of the chromosomal region having the group of genes thus detected and analyzing the group of genes amplified and deleted, myeloma can be diagnosed.

As described above, the amplification and deletion of the chromosomal region in myeloma are analyzed by use of the MCG cancer array, and thus a group of genes having amplified and deleted can be identified. Based on the results, it is possible to understand the state of each cancer. To describe more specifically, it is possible to determine whether a tumor is benign, intermediate or malignant. In the case of a malignant tumor, it is possible to provide important findings to determine the grade of the cancer. It is further possible to provide data for efficient chemotherapy performed after a cancerous foci is surgically removed.

It is possible and preferable to simultaneously detect deletion of a chromosome and suppression of expression by monitoring the gene expression by a real time RT-PCR method or a DNA chip method in a deletion cancer gene group.

B. Suppression/Treatment Means for a Cancer by a Cancer-Associated Gene

The suppression/treatment means for a cancer provided by the present invention are roughly divided into two groups. One (1) is a method of suppressing the cancer cell (hereinafter referred to as “suppression/treatment means 1”) by introducing a gene whose deletion is associated with canceration of a cell (called as a deletion cancer gene) into a cancer cell. The other (2) is a method of suppressing the cancer cell (hereinafter referred to as “suppression/treatment means 2”) by applying a nucleic acid antagonizing against a transcriptional product of a gene whose amplification is associated with canceration of a cell (called as an amplification cancer gene) to a cancer cell.

(1) Suppression/Treatment Means 1

Of the deletion cancer genes mentioned above, many of the genes in the chromosomal region exhibiting a homozygous deletion are detected to fall within the category of a cancer suppressor gene. Of them, a gene suppressing proliferation of target cancer cells or a gene inducing apoptosis of cancer to death can be introduced into a cancer cell by use of a Sendai virus vector or adenovirus vector. In a gene therapy using these virus vectors, as a promoter for the homozygous deletion gene to be expressed, a promoter highly expressed in a cancer tissue but not highly expressed in a normal tissue, such as human CXCR4 promoter (Zhu Z B, Makhija S K, Lu B, Wang M, Kaliberova L, Liu B, Rivera A A, Nettelbeck D M, Mahasreshti P J, Leath C A, Yamaoto M, Alvarez R D, Curiel D T: Transcriptional targeting of adenoviral vector through the CXCR4 tumor—specific promoter, Gene ther., 11, 645-648, 2004) and Survivin promoter are preferably used. Each of these recombinant viruses can be combined with a ribosome to form a composite, which may be introduced into a cancer tissue. Alternatively, it can be introduced in the form of naked DNA into a cancer tissue.

Using a viral vector and a promoter as mentioned above, each cancer therapy can be made by selecting a gene from following candidate genes:
EPS15 gene localized in 1p32, PGR, cIAP1, MMP1, DYNEIN gene localized in 11q22, YAP1 localized in 11q22.1, MMP7 gene localized in 11q21-q22, ETS1 gene localized in 11q23.3, FLI1 gene localized in 11q24, IGHG1 gene localized in 14q32.33, and TSPY gene localized in Ypter-p11.2 for myeloma.

CDKN2A(p16) gene is a cyclin dependent kinase inhibitor located in a chromosome 9p21 and considered as a cancer suppressor gene. P16 protein, when it binds to CDK4 kinase, is suppressed in its activity, thereby suppressing cell cycle progression. The CDKN2A(p16) gene is deleted in a wide variety of cancers such as acellular esophageal carcinoma, malignant glioma, gastric carcinoma, pancreatic carcinoma and thyroid carcinoma. MTAP is a gene encoding 5′-methylthioadenosinephosphorylase, which is the first enzyme of a methionine salvage pathway and considered as a cancer suppressor gene. The product of the methionine salvage pathway inhibits the activity of ornithine decarboxylase highly expressed in cancer. RIZ is a gene encoding an RB interacting Zinc Finger protein found in leukemia and belongs to Nuclear protein methyltransferase superfamily. DBCCR1 is found as a gene deleted in chromosome 1 of the bladder carcinoma and considered as a cancer suppressor gene. TEK is an angiopoietin-1 receptor, which is otherwise designated as Tie-2. When TEK is phosphorylated by tyrosine kinase, angiogenesis is induced. CDH23 is cadherin related 23 gene, belongs in the cadherin superfamily, and is a glycoprotein associated with calcium dependent cell adhesion. CXADR gene encodes receptors of coxsachie virus and adenovirus. cIAP1 gene encodes an apoptosis inhibitor. FLI1 gene is classified into an ETS transcription factor. TSPY gene is present in human Y chromosome and encodes a testis specific protein. LRP1B is abbreviation of lipoprotein receptor-related protein 1B, which is a cellular membrane receptor using urokinase and a plasminogen activator, etc., as a ligand, and is considered as a cancer suppressor gene. DEC1 refers to “deleted in esophageal cancer 1” and loss of heterozygosity is frequently detected in esophageal carcinoma and squamous cell carcinoma of the bladder, lung and head and neck portion. MMP1 and MMP7 are matrix metalloproteinase and enzymes involved in vascularization. SMAD4 gene is a cancer suppressor gene whose deletion is found in pancreatic carcinoma and encodes a protein that is activated by a receptor and transferred to a nucleus to derive a transcriptional activation activity. ETS1 is a transcription factor and derives angiopoietin-2 gene, etc. RB1 is a retinoblastoma gene and a cancer suppressor gene.

A virus vector is prepared by integrating a gene as mentioned above downstream of a promoter highly expressed in a cancer tissue, and is then introduced into the cancer tissue of a cancer patient. The gene is allowed to express, thereby reducing cancer in size and inhibiting metastasis. In this way, recurrence of cancer after cancer is excised out can be prevented.

(2) Suppression/Therapeutic Means 2

Of the amplification cancer genes found above, a group of genes present in the chromosome, amplified 4-fold or more than that of a normal cell, are shown in Table 1.

TABLE 1

| Type of | | | | | | |
|---|---|---|---|---|---|---|
| cancer cell | Name of amplified gene | | | | | |
| Myeloma | ABL | CAN | KRAS2 | ZNF285 | D17S128 | BCL2 |
| | KRAG | PTHLH | NCOR1 | FVT1 | PI5 | |

When these groups of genes are compared to those of a normal cell, the number of genome copies in chromosomes 1 to 22 increases to 8 or more, and that in X and Y chromosomes increases 4 or more. The transcriptional product of a highly expressed gene is decomposed by adding the small interference RNA corresponding to the transcriptional product (mRNA) in accordance with an RNAi (RNA interference) method. In this manner, cancer can be treated. Design and synthesis of siRNA and the transfection of siRNA to a cell, confirmation of the effect of RNAi can be performed by conventional methods with reference to, for example, Takara Bio RNAi Book, “Experimentation protocol” (published by Takara Bio Inc., Shiga prefecture). Examples of siRNA to be used herein include Hairpin siRNA, which can be expressed by using an siRNA oligonucleotide and a pSilencer vector (manufactured by Funakoshi Co., Ltd., Tokyo).

On the other hand, mRNA of a cancer gene amplified and excessively expressed in a cancer can be knocked out by use of an antisense oligonucleotide. In this case, s-oligonucleotide is preferably used to inhibit amplification of a cancer cell since it has a good intracellular stability compared to a general oligonucleotide. SiRNA, Hairpin siRNA and s-oligonucleotide, which are found to be effective by use of a cancer cell, can be evaluated in a nude mouse having a cancer cell transplanted therein.

In this case, it is preferable to construct a delivery system such that these RNA can be accumulated in a cancer tissue.

### EXAMPLES

### Example 1

### Preparation of “MCG Cancer Array”

Based on the search for genome database website of the National Cancer for Biotechnology and University of California, Santa Cruz Biotechnology as well as BLAST search of DNA screened, BAC/PAC clones having an extremely important gene for canceration and amplification of a cancer cell or having a sequence tagged site marker were selected.

BAC and PAC DNA was digested with Dpnl, RsaI, and HaeIII, and thereafter ligated with adaptor DNA. PCR was performed twice using a primer having the sequence of the adaptor. One of the two ends of the primers has the 5′ end aminated. This process is called an inexhaustible process and DNA thus obtained is defined as inexhaustible DNA. The inexhaustible DNA is placed in an ink-jet type spotter (GENESHOT, NGK Insulators, Ltd., Nagoya) and covalently printed, in duplicate, onto an oligo DNA micro array (manufactured by Matsunami Glass, Osaka).

### Example 2

### Collective Analysis of a Cancer-Associated Gene in Myeloma by Use of the MCG Cancer Array

Using the “MCG cancer array,” a gene amplified and deleted was checked with respect to myeloma cell lines. A gene amplified and having a Ratio value of 1.32 or more was checked. As a result, BCL9, MCL1, AF1Q, MUC1, DAP3, ARHGEF2, PMF1, BRAL1, PRCC, TRK, NTRK1, CRP, ATF6, PBX1, ABL2, LAMC2, and TP were found (Table 2). The amplification of these genes was detected with a high frequency of 63 to 82% of myeloma cell lines tested herein.

TABLE 2
Name of gene amplified and having a Ratio
value of 1.32 or more in myeloma cell

| Chromosomal region | Name of amplified gene | % |
|---|---|---|
| 1q21 | BCL9 | 63.0 |
| 1q21 | MCL1 | 70.4 |
| 1q21 | AF1Q | 81.5 |
| 1q21 | MUC1 | 81.5 |
| 1q21 | DAP3 | 48.1 |
| 1q21 | DAP3 | 55.6 |
| 1q21 | ARHGEF2 | 81.5 |
| 1q23.1 | PMF1 | 55.6 |
| 1q23.1 | BRAL1 | 74.1 |
| 1q21 | PRCC | 81.5 |
| 1q23-q24 | TRK | 63.0 |
| 1q21 | NTRK1 | 70.4 |
| 1q21-q23 | CRP | 63.0 |
| 1q23.3 | ATF6 | 44.4 |
| 1q23 | PBX1 | 55.6 |
| 1q24-q25 | ABL2 | 66.7 |
| 1q24-q25 | ABL2 | 70.4 |
| 1q25-q31 | LAMC2 | 63.0 |
| 1q25 | TPR | 59.3 |

As a gene having a Ratio value of not less than 4 in a myeloma cell, that is, a gene having not less than 4-fold amplification as compared to that in a normal cell, ABL, CAN, KRAS2, KRAG, PTHLH, NCOR1, ZNF287, D17S128, BCL2, FVT1, and PI5 genes were found (Table 3.

TABLE 3
Name of gene having a Ratio value
of not less than 4 in myeloma cell

| Chromosomal region | Name of amplified gene |
|---|---|
| 9q34 | ABL1 |
| 9q34.1 | CAN |
| 12p12.1 | KRAS2 |
| 12p11.2 | KRAG |
| 12p12.1-p11.2 | PTHLH |
| 17p11.2 | NCOR1 |
| 17p11.2 | ZNF287 |
| 17p11.2 | D17S1280 |
| 18q22 | BCL2 |
| 18q21.3 | FVT1 |
| 18q21.3 | PI5 |

Next, as a gene having a Ratio value reduced to 0.75 or less in a myeloma cell, that is, a gene determined as a heterozygote, TGFβR3, ABCD3, ZNF198, FGF9, FLT3, FLT1, BRCA2, FKHR, TPT1, LCP1, RB1, DACH, PIBF1, KLF12, GPC5, GPC5 (2), GPC6, ABCC4, RAP2A, FGF14, ERCC5, EFNB2, ING1, TFDP1, GAS6, D13S327, TEP1, MMP14, BCL2L2, FKHL1, MBIP, HNF3A, SSTR1, PNN (DRS), CDKN3, RBBP1, DAAM1, MNAT1, HIFIA, ESR2, MAX, EIF2S1, RAD51L1, IGHG1, HSXIAPAF1, MN1, and TSPY were found (Table 4). A heterozygous deletion of these genes was detected with a high frequency of 40 to 89% of myeloma cell lines tested herein.

TABLE 4
Name of gene having a Ratio value
reduced to 0.75 in myeloma cell

| Chromosomal region | Name of deleted gene | % |
|---|---|---|
| 1p33-p32 | TGFβR3 | 51.9% |
| 1p21.3 | ABCD3 | 51.9% |
| 13q11-q12 | ZNF198 | 85.2% |
| 13q11-q12 | FGF9 | 81.5% |
| 13q12 | FLT3 | 81.5% |
| 13q12 | FLT1 | 70.4% |
| 13q12-13 | BRCA2 | 74.1% |
| 13q14.1 | FKHR | 66.7% |
| 13q12-q14 | TPT1 | 70.4% |
| 13q14.1-q14.3 | LCP1 | 66.7% |
| 13q14 | RB1 | 66.7% |
| 13q22 | DACH | 63.0% |
| 13q22.1 | PIBF1 | 66.7% |
| 13q22.1 | KLF12 | 70.4% |
| 13q32 | GPC5 | 48.1% |
| 13q32 | GPC5(2) | 40.7% |
| 13q32 | GPC6 | 51.9% |
| 13q32 | ABCC4 | 85.2% |
| 13q34 | RAP2A | 85.2% |
| 13q34 | FGF14 | 81.5% |
| 13q22 | ERCC5 | 77.8% |
| 13q33 | EFNB2 | 85.2% |
| 13q34 | ING1 | 66.7% |
| 13q34 | TFDP1 | 63.0% |
| 13q34 | GAS6 | 70.4% |
| 13qte1 | D13S327 | 81.5% |
| 14q11.2 | TEP1 | 70.4% |
| 14q11-q12 | MMP14 | 66.7% |
| 14q11.2 | BCL2L2 | 77.8% |
| 14q13 | FKHL1 | 59.3% |
| 14q12 | MBIP | 55.6% |
| 14q12 | HNF3A | 63.0% |
| 14q13 | SSTR1 | 51.9% |
| 14q13 | PNN(DRS) | 74.1% |
| 14q22 | CDKN3 | 63.0% |
| 14q22.3 | RBBP1 | 51.9% |
| 14q23.1 | DAAM1 | 63.0% |
| 14q23 | MNAT1 | 51.9% |
| 14q24 | HIF1A | 51.9% |
| 14q | ESR2 | 48.1% |
| 14q23 | MAX | 59.3% |
| 14q23.3 | EIF2S1 | 40.7% |
| 14q24 | RAD51L1 | 55.6% |
| 14q32.33 | IGHG1 | 88.9% |
| 17p13.1 | HSXIAPAF1 | 55.6% |
| 22q12.3-qter | MN1 | 51.9% |
| Ypter-pll.2 | TSPY | 85.2% |

Furthermore, as a gene having a Ratio value of 0.25 or less, that is, a gene in which a homozygous deletion was detected, EPS15, PGR, YAP1, cIAP1, MMP7, MMP1, DYNEIN, ETS1, FLI1, 1GHG1, and TSPY genes were found (Table 5). A group of genes having heterozygote and homozygote significantly decreases in expression level, which may possibly be a cause of cancer.

In particular, a gene having a homozygous deletion is a cancer suppressor gene. The deletion taking place in the group of genes plays an important role in inducing canceration.

TABLE 5
Name of gene having a Ratio value reduced
to 0.25 or less in myeloma cell

| Chromosomal region | Name of amplified gene |
|---|---|
| 1p32 | EPS15 |
| 11q22 | PGR |
| 11q22.1 | YAP1 |
| 11q22 | cIAP1 |
| 11q21-q22 | MMP7 |
| 11q22 | MMP1 |
| 11q22 | DYNEIN |
| 11q23.3 | ETS1 |
| 11q24 | FLI1 |
| 14q32.33 | IGHG1 |
| Ypter-p11.2 | TSPY |

### INDUSTRIAL APPLICABILITY

According to the present invention, a cancer-associated gene to be used as an index for detecting canceration of a cell and degree of malignancy of cancer was found, and a method of detecting cancer using the cancer-associated gene as an index was provided, and furthermore a suppression/therapeutic method of cancer using the cancer-associated gene as essential part was provided.

1. A method for detecting myeloma, wherein canceration of a specimen is detected based on an index of not less than 1.5 fold amplification of at least one gene selected from the group consisting of
BCL9 gene, MCL1 gene, AF1Q gene, MUC1 gene, DAP3 gene, ARHGEF2 gene, PMF1 gene, BRAL1 gene, PRCC gene, TRK gene, NTRK1 gene, CRP gene, ATF6 gene, PBX1 gene, ABL2 gene, LAMC2 gene, TP gene, ABL gene, CAN gene, KRAS2 gene, KRAG gene, PTHLH gene, NCOR1 gene, ZNF287 gene, D17S128 gene, BCL2 gene, FVT1 gene, and PI5 gene;
in the specimen in comparison with a normal cell.

2. The method according to claim 1, wherein canceration of a specimen is detected based on an index of not less than 4 fold amplification of at least one gene selected from the group consisting of
ABL gene, CAN gene, KRAS2 gene, KRAG gene, PTHLH gene, NCOR1 gene, ZNF287 gene, D17S128 gene, BCL2 gene, FVT1 gene, and PI5 gene;
in the specimen in comparison with a normal cell.

3. A method for detecting myeloma, wherein canceration of a specimen is detected based on an index of a heterozygous deletion of at least one gene selected from the group consisting of
TGFβR3 gene, ABCD3 gene, ZNF198 gene, FGF9 gene, FLT1 gene, BRCA2 gene, FKHR gene, TPT1 gene, LCP1 gene, RB1 gene, DACH gene, PIBF1 gene, KLF12 gene, GPC5 gene, GPC5 (2) gene, GPC6 gene, ABCC4 gene, RAP2A gene, FGF14 gene, ERCC5 gene, EFNB2 gene, ING1 gene, TFDP1 gene, GAS6 gene, D13S327 gene, TEP1 gene, MMP14 gene, BCL2L2 gene, FKHL1 gene, MBIP gene, HNF3A gene, SSTR1 gene, PNN (DRS) gene, CDKN3 gene, RBBP1 gene, DAAM1 gene, MNAT1 gene, HIF1A gene, ESR2 gene, MAX gene, EIF2S1 gene, RAD51L1 gene, IGHG1 gene, HSXIAPAF1 gene, MN1 gene, TSPY gene, EPS15 gene, PGR gene, YAP1 gene, cIAP1 gene, MMP7 gene, DYNEIN gene, ETS1 gene, and FLI1 gene.
in the specimen.

4. A method for detecting myeloma, wherein canceration of a specimen is detected based on an index of a homozygous deletion of at least one gene selected from the group consisting of
EPS15 gene, PGR gene, YAP1 gene, cIAP1 gene, MMP7 gene, DYNEIN gene, ETS1 gene, FLI1 gene, IGHG1 gene, and TSPY gene;
in the specimen.

5. The detection method according to claim 1, wherein the detection is performed by a CGH method, DNA chip method, quantitative PCR method or real time RT-PCR method.

6. The detection method according to claim 1, wherein the detection is performed by a CGH method or DNA chip method and a plurality of types of DNA fragments to be fixed onto the detection substrate are genomic DNA, cDNA or synthetic oligonucleotides.

7. The detection method according to claim 1, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

8. The detection method according to claim 3, wherein the detection is performed by a CGH method, DNA chip method, quantitative PCR method or real time RT-PCR method.

9. The detection method according to claim 3, wherein the detection is performed by a CGH method or DNA chip method and a plurality of types of DNA fragments to be fixed onto the detection substrate are genomic DNA, cDNA or synthetic oligonucleotides.

10. The detection method according to claim 3, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

11. The detection method according to claim 4, wherein the detection is performed by a CGH method, DNA chip method, quantitative PCR method or real time RT-PCR method.

12. The detection method according to claim 4, wherein the detection is performed by a CGH method or DNA chip method and a plurality of types of DNA fragments to be fixed onto the detection substrate are genomic DNA, cDNA or synthetic oligonucleotides.

13. The detection method according to claim 4, wherein the detection is performed by a CGH method, and a plurality of types of DNA fragments to be fixed onto the detection substrate are genomic DNA, and the genomic DNA is a gene amplification product of BAC DNA, YAC DNA or PAC DNA.

14. A method for suppressing a myeloma cell, which comprises introducing a gene, whose deletion is involved in canceration of a myeloma cell, into a myeloma cell.

15. A method for suppressing a myeloma cell, which comprises introducing at least one gene selected from the group consisting of EPS15 gene, PGR gene, cIAP1 gene, DYNEIN gene, YAP1 gene, MMP7 gene, ETS1 gene, FLI1 gene, IGHG1 gene and TSPY gene into a myeloma cell.

16. A method of suppressing a myeloma cell, which comprises applying, to a myeloma cell, a nucleic acid antagonizing a transcriptional product of a gene whose amplification is involved in canceration of the myeloma cell.

17. A method of suppressing a myeloma cell, which comprises applying, to a myeloma cell, a nucleic acid antagonizing a transcriptional product of at least one gene selected from the group consisting of ABL gene, CAN gene, KRAS2 gene, ZNF285 gene, D17S128 gene, BCL2 gene, KRAG gene, PTHLH gene, NCOR1 gene, FVT1 gene and PI5 gene.

18. The method according to claim 16, wherein the nucleic acid antagonizing a transcriptional product of a gene is small interference RNA against a transcriptional poroduct mRNA, or an antisense oligonucleotide of the mRNA.
